(19) United States
(12) Patent Application Publication
(10) Pub. No.: US 2021/0196497 A1
(43) Pub. Date: Jul. 1, 2021

(54) Ankle Inversion Sprain Protection Device

(71) Applicant: Tung-Ju Chuang, New Taipei City (TW)

(72) Inventor: Tung-Ju Chuang, New Taipei City (TW)

(21) Appl. No.: 16/871,028

(22) Filed: May 10, 2020

(30) Foreign Application Priority Data
Dec. 27, 2019 (TW) 108217383

(51) Int. Cl. A61F 5/01; A43B 7/20

(52) U.S. Cl. CPC A61F 5/0111; A43B 7/20

(57) ABSTRACT

An ankle inversion sprain protection device, adapted for various shoe bodies, includes a first lacing element, fastened at the lateral bottom position in the shoe, and a second lacing element, extended outwardly from a top end of the first lacing element so as for being embraced above an ankle, such that when the ankle is overly turned inwards, the ankle will be held from the outer side by the second lacing element and therefore be protected from inversion sprain. The present invention is different from conventional ankle guard and provides a more effective protection for the ankle in a comfortable and convenient manner to the user.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to a technology of an ankle inversion sprain protection device, and more particularly to a device to prevent an ankle from sprain through holding it from an outer side thereof by means of a lacing element while the ankle is overly turned inwardly. The ankle inversion sprain protection device also provides a comfortable and convenient experience to the user.

### Description of Related Arts

Ankle inversion sprain is commonly seen in the athletic fields, especially in the sport of basketball, even for the NBA players who have the best protection equipment. It follows that conventional protection devices, such as ankle guards or sports shoes, are incapable of providing effective inversion sprain protection.

The most common ankle protection device in the market is ankle guard and, unfortunately, it fails to prevent inversion sprain. Among all reasons, it is because that the entire external shape of human body from the ankle to the sole is curvy and smooth, rendering the ankle guard difficult to grasp the ankle firmly. Consequently, the ankle guard will usually loose gradually and lose its protection over time under intense activity.

All types of ankle guard are secured through wrapping the sole bottom, which is likely to cause bumpy feelings on the user's sole. As a result, the user often feels uncomfortable or pain over time during exercise. In addition, there are so-called high level ankle guards in the market, which are costly, structurally complex, operationally time-consuming, and unfriendly to the users.

Moreover, high-top sneakers, which are commonly seen in the market, are usually mistakenly believed to be capable of protecting the ankles from sprain because they cover above the ankles. Nevertheless, high-top sneakers, according to numerous studies, have no significant function in preventing sprain. This can further be proven from the fact that most NBA players do not wear high-top sneakers. The reason is that even if the shoelaces are tied up tightly, when the ankle is turned inwards, a gap will still occur between the outer side of the ankle and the shoe body of the high-top sneakers, rendering the ankle unprotected and further pushed down, which leads to more serious sprain.

### SUMMARY OF THE PRESENT INVENTION

An object of the present invention is to provide an ankle inversion sprain protection device, which is different from conventional ankle guard and is capable of more effectively protecting the ankles in a comfortable and convenient manner to the user.

In order to achieve the above and other objects, the present invention provides an ankle inversion sprain protection device, adapted for various shoe bodies, which comprises:

a first lacing element, fastened at the lateral bottom position in the shoe, and

a second lacing element, extended outwardly from a top end of the first lacing element so as for being embraced above an ankle, such that when the ankle is overly turned inwards, the ankle will be held from the outer side by the second lacing element and therefore be protected from inversion sprain.

According to a preferred embodiment, the second lacing element is positioned through hook and loop fasteners or other means.

According to a preferred embodiment, the first lacing element is fastened at the lateral bottom position in the shoe through stitching, adhering, or other means.

According to a preferred embodiment, the first lacing element is adhered on the bottom of the inner side of the shoe body by the user and covered by the insole of the shoe body, such that the user will not feel bumpy when stamping.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view illustrating a right side for a right foot of an ankle inversion sprain protection device according to a preferred embodiment of the present invention.

FIG. 2 is a perspective view illustrating a left side for a right foot of the ankle inversion sprain protection device according to the above preferred embodiment of the present invention.

### DETAILED DESCRIPTION OF THE PREFERRED EMBODIMENT

The following disclosure is to illustrate implementations of the present invention through specific embodiments, so that those skilled in the art can easily understand other advantages and functions of the present invention through the disclosure.

Referring to the figures, embodiments of the present invention will be illustrated. It is worth mentioning that the figures of the present invention are only to provide simple schematic illustration for the perspectives of basic concepts of the present invention, rather than to depict based on the quantities, shapes, and dimensions of the elements that will actually be implemented. Therefore, the forms, quantities, and proportions of the elements that will actually be implemented may change based on the actual demand, rather than be limited by the figures.

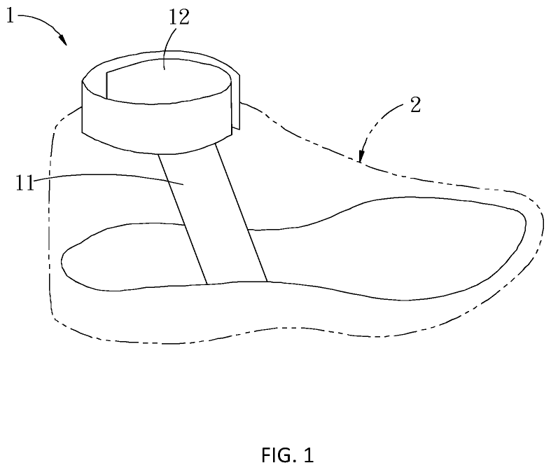

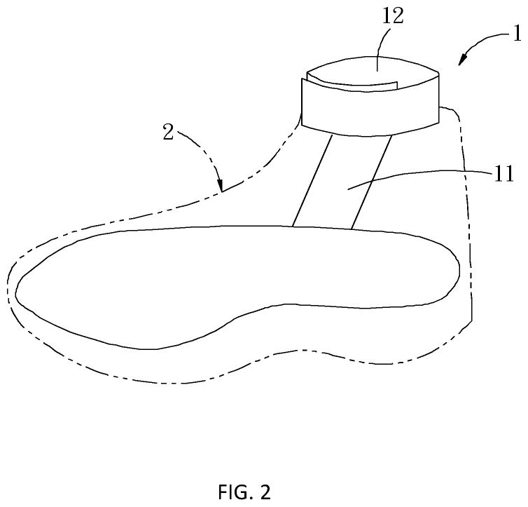

FIGS. 1 and 2 are perspective views illustrating the right side and left side for a right foot of an ankle inversion sprain protection device according to a preferred embodiment of the present invention. Referring to the figures, the ankle inversion sprain protection device 1 of the present invention is adapted for various shoe bodies 2. The ankle inversion sprain protection device 1 comprises a first lacing element 11 and a second lacing element 12. The first lacing element 11 has a first end and a second end configured to be fastened at a lateral bottom position in the shoe body 2. The second lacing element 12 is extended outwardly from the first end of the first lacing element 11 to form a circular element shaped and configured for being circularly embraced above an ankle of the wearer of the shoe body 2, such that when the ankle is overly turned inwards, the ankle will be held from the outer side by the second lacing element 12 and therefore be protected from inversion sprain.

According to a preferred embodiment, the second lacing element 12 has two ends which are detachably connected through a pair of hook and loop fasteners or other means, provided at the two ends respectively to form the circular element. The first lacing element 11 is fastened at the lateral bottom position in the shoe through stitching, adhering, or other means. According to another preferred embodiment, the first lacing element 11 may also be adhered on the bottom of the inner side of the shoe body 2 by the user and covered by the insole of the shoe body 2, such that the user will not feel bumpy when stamping.

Specifically speaking, the ankle inversion sprain protection device 1 of the present invention may be directly seamed and secured on the bottom of various shoe bodies or independently provided for the user to place into the shoe body 2 and cover the insole thereon. In other words, it may be integrated with various types of shoe body or be arranged on the bottom of the shoe, which provides flexible implementation and enhances the overall utility and user-friendliness thereof.

Most of the conventional ankle protection devices in the market gradually become loose and lose their protection over time under intense activity. Besides, they usually make the user's foot feel bumpy and uncomfortable. As for high-top sneakers, when an ankle turns inwards, there will be gap between the outer side of the ankle and the shoe body, rendering the ankle unprotected and further pushed down, which leads to more serious sprain. Through the design of the structure of the first lacing element and the second lacing element, the ankle inversion sprain protection device of the present invention is capable of preventing the above drawbacks and enhancing the protection effectively in a comfortable and convenient manner to the user.

Although the above descriptions and figures have disclosed preferred embodiments of the present invention, additions, modifications, and alternatives may also apply to the preferred embodiment of the present invention without exceeding the spirit and scope of the principles of the present invention defined by the appended claims. Person skilled in the art should be able to understand that the present invention may also be implemented in modifications of various forms, structures, arrangements, ratios, materials, elements, and units. Therefore, the above mentioned embodiments are to illustrate the present invention and shall not be used to limit the scope of the present invention. The scope of the present invention shall be defined by the appended claims and cover all the equivalent, which shall not be limited by the above description.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. An ankle inversion sprain protection device adapted for being arranged in a shoe body, comprising:
a first lacing element having a first end and a second end configured for being fastened at a lateral bottom position in the shoe body; and
a second lacing element, extended outwardly from said first end of said first lacing element to form a circular element shaped and configured for being circularly embraced above an ankle of a wearer of the shoe body, such that when the ankle is overly turned inwards, the ankle will be held from the outer side by said second lacing element and therefore be protected from inversion sprain.

2. The ankle inversion sprain protection device, as recited in claim 1, wherein said second lacing element has two ends which are able to be detachably connected to form said circular element through a fastening means which includes at least a pair of hook and loop fasteners provided at the two ends respectively.

3. An ankle inversion sprain protection shoe, comprising:
a shoe body;
a first lacing element having a first end and a second end fastened at a lateral bottom position in said shoe body; and
a second lacing element, extended outwardly from said first end of said first lacing element to form a circular element shaped and configured for being embraced above an ankle of a wearer of said shoe body, such that when the ankle is overly turned inwards, the ankle will be held from the outer side by said second lacing element and therefore be protected from inversion sprain.

4. The ankle inversion sprain protection shoe, as recited in claim 3, wherein said second lacing element has two ends which are able to be detachably connected to form said circular element through a fastening means which includes at least a pair of hook and loop fasteners provided at the two ends respectively.

5. The ankle inversion sprain protection shoe, as recited in claim 3, wherein said second end of said first lacing element is fastened at said lateral bottom position in said shoe.

6. The ankle inversion sprain protection shoe, as recited in claim 5, wherein said second end of said first lacing element is fastened at said lateral bottom position in said shoe through a means selected from stitching and adhering.

7. The ankle inversion sprain protection shoe, as recited in claim 4, wherein said second end of said first lacing element is fastened at said lateral bottom position in said shoe.

8. The ankle inversion sprain protection shoe, as recited in claim 7, wherein said second end of said first lacing element is fastened at said lateral bottom position in said shoe through a means selected from stitching and adhering.

9. The ankle inversion sprain protection shoe, as recited in claim 3, wherein said second end of said first lacing element is adhered on a bottom of an inner side of said shoe body and covered by an insole of said shoe body.

10. The ankle inversion sprain protection shoe, as recited in claim 4, wherein said second end of said first lacing element is adhered on a bottom of an inner side of said shoe body and covered by an insole of said shoe body.
